(19) United States
(12) Patent Application Publication
(10) Pub. No.: US 2026/0263355 A1
(43) Pub. Date: Sep. 10, 2026

(54) DRUG-LOADED PAD FOR IMPLANTING ON LYMPHATIC TISSUE

(71) Applicant: TOLYMPH INC., Richmond Hill (CA)

(72) Inventor: Jiang LIU, Ajax (CA)

(21) Appl. No.: 19/164,739

(22) PCT Filed: Feb. 10, 2025
(86) PCT No.: PCT/CA2025/050171

§ 371 Date: Sep. 12, 2025

Related U.S. Application Data
(60) Provisional application No. 63/552,146, filed on Feb. 11, 2024.

(51) Int. Cl. A61K 9/00; A61K 9/16; A61K 9/70; A61K 31/337; A61P 35/00

(52) U.S. Cl. CPC A61K 9/0024; A61K 9/1647; A61K 9/7007; A61K 31/337; A61P 35/00

(57) ABSTRACT

A drug-loaded pad that is implanted onto target lymphatic tissue and in which the drug is released therefrom. The drug may be paclitaxel or docetaxel or cabazitaxel. The drug-loaded pad contains a therapeutically effective amount of the drug. The drug-loaded pad comprises a bulk matrix and drug particles carried by the bulk matrix. The bulk matrix comprises a biodegradable polymer. The drug-loaded pad may have a thickness of 2-8 mm. Also disclosed is a method of treating cancer in a patient comprising implanting the drug-loaded pad onto a target lymphatic tissue.

### TECHNICAL FIELD

This invention relates to drug-releasing implants for the treatment of cancer.

### BACKGROUND

Metastatic cancer is the spread of cancer cells from the primary tumor to distant sites. The lymphatic system plays a crucial role in the metastatic process, serving as a conduit for cancer cells to disseminate throughout the body. Targeting the lymphatic system offers a promising approach to intercept and eliminate metastatic cancer cells before they establish secondary tumors. Conventional systemic drug delivery methods, such as intravenous administration of chemotherapeutics, face several limitations in treating lymphatic metastases. In particular, there is poor lymphatic penetration of drugs, insufficient drug concentrations at lymphatic metastatic sites, and limited residence time in the lymphatic system.

To address the deficiencies of systemic drug delivery, implants for localized drug delivery have been developed that focus on delivery to specific tissue sites. However, existing drug delivery implants have limitations including inability to achieve sustained lymphatic drug concentrations, poor integration with lymphatic vessels, risk of implant migration, limited drug payload capacity, and lack of controlled release mechanisms specific to the lymphatic environment.

There is an urgent need for innovative drug delivery technologies that can deliver drugs directly to the lymphatic system, maximizing their efficacy and minimizing systemic toxicity. In particular, there remains an unmet need for an implantable drug delivery system that can provide controlled long-term release of drugs, maintain therapeutic drug concentrations within the lymphatic system, minimize systemic drug exposure and associated toxicities, and allow for minimally invasive placement.

### SUMMARY

This invention provides a drug-loaded pad that is applied onto a target site and in which the drug is released therefrom. The drug may be a taxane-type chemotherapeutic drug such as paclitaxel, docetaxel, or cabazitaxel. Other drugs that could be used include those listed in patent literature US 2021/0077665 (by Jiang Liu et al), which is incorporated by reference herein.

The drug-loaded pad has a generally flat profile and may have any suitable shape, such as round, oval, square, ring, triangle, rectangular strip, etc. The drug-loaded pad may have any suitable dimensions. For example, the drug-loaded pad may have a thickness of 2-8 mm, and in some cases, 3-6 mm. The total mass of the drug-loaded pad could be 10-50 mg or 10-258 mg. Other ranges are also possible, such as ≥10 mg. The drug-loaded pad may be flexible so that it can conform and adhere to the target site of implantation.

The drug-loaded pad comprises a bulk matrix and drug particles carried by the bulk matrix. The bulk matrix comprises one or more biodegradable polymers. Examples of such include poly(lactic-co-glycolic acid) (PLGA), poly(lactic acid) (PLA), polycaprolactone (PCL), polysorbate, cellulose, chitosan, alginate, collagen, gelatin, etc. Further examples of biodegradable polymers that could be used are listed in patent literature US 2021/0077665 (by Jiang Liu et al), which is incorporated by reference herein. The bulk matrix may further comprise one or more other excipient ingredients such as fillers, solubilizing agents, preservatives, penetration enhancers, binders, disintegrants, diluents, etc. One particular excipient ingredient could be polysorbate (such as the Tween family of surfactant materials). Further examples of excipient ingredients that could be used are listed in patent literature US 2021/0077665 (by Jiang Liu et al), which is incorporated by reference herein. The bulk matrix may constitute 30-75 wt % of the drug-loaded pad, and in some cases, 45-60 wt %.

The drug particles comprise the drug (e.g. paclitaxel or docetaxel or cabazitaxel). The drug particles further comprise one or more biodegradable polymers. The drug particles may further comprise one or more other excipient ingredients. The drug particles are embedded or dispersed within the bulk matrix. The drug particles may have any suitable size for absorption by the lymphatic tissue. In some embodiments, the drug particles have a size in the range of 0.5-10 μm, and in some cases, 0.5-7 μm. In some embodiments, the drug particles have a size in the range of 10-500 nm.

The drug-loaded pad may have any therapeutically effective amount of the drug. The drug may constitute 3-40 wt % of the drug-loaded pad; in some cases, 5-25 wt %; and in some cases, 1-15 wt %. The total amount of drug contained in the drug-loaded pad may be ≤12 mg, or ≤8 mg, or ≤6 mg, or ≤5.5 mg. To define a lower limit, the amount of drug contained in the drug-loaded pad may be ≥0.2 mg or ≥0.5 mg or ≥0.7 mg. As such, example ranges for the amount of drug contained in the drug-loaded pad with these parameters include the following: 0.2-12 mg, 0.5-8 mg, 0.7-6 mg, or 0.2-5.5 mg.

Treatment Method. In another aspect, this invention is a method of treating cancer. The drug-loaded pad is implanted onto a target lymphatic tissue. As used herein, “lymphatic tissue” means a part of the lymphatic system such as lymph nodes, lymphatic vessels, or lymphoid tissue. Be aware that even in situations where lymph nodes are surgically removed (leaving a lymph node fossa), lymphoid or paranode tissue may still remain at the site, which may be the target lymphatic tissue for this invention.

One or more drug-loaded pads may be implanted onto the target lymphatic tissue. The drug-loaded pad releases the drug in a spatially and temporally controlled manner. The drug particles are released from the drug-loaded pad and are taken up by lymphatics and drained towards regional and remote lymph nodes. The drug particles are trapped in the lymph nodes because of their size and composition (spatial control). The drug particles then further disintegrate to release the drug payload at the targeted lymphatic tissue (temporal control). The total amount of drug given to the patient via the drug-loaded pad may be in the range of 2-15 mg; in some cases, 2-11 mg; and in some cases, 3-9 mg. In other embodiments, the total amount of drug given to the patient via the drug-loaded pad may be in the range of 1-60 mg; and in some cases, 3-21 mg. These are calculated by dose extrapolation to humans from the experimental work performed on intrapleural implantation in Beagle dogs (see below). Dose extrapolation may vary in different body compartments accordingly. The drug-loaded pad can be multiplied during the treatment according to the dose required for patients (e.g. implanting several drug-loaded pads on the same target lymphatic tissue or implanting multiple pads to various lymphatic tissues).

The drug-loaded pad may be applied to the target lymphatic tissue without the need for sutures or glue. The drug-loaded pad may absorb moisture and self-adhere to the target site. The drug-loaded pad may be shapeable so that it conforms to the target lymphatic tissue.

The drug-loaded pad may be implanted as part of a surgical procedure before or during the removal of a malignant tumor. The drug-loaded pad may be implanted intra-operatively during the surgical procedure. The surgical procedure may be open surgery or minimally invasive surgery (MIS). For MIS, the surgery is performed endoscopically (e.g. by laparoscopy, thoracoscopy, or mediastinoscopy etc.) and the drug-loaded pad implanted via the operating port or channel. The surgical procedure may be tumor resection surgery or diagnostic (e.g. exploratory, lymph node biopsy, etc.). The cancer may have lymphatic metastasis from the site of a primary tumor. In this context, the tumor resection surgery may be for resection of the primary tumor and/or metastatic or suspicious lymph nodes. The target lymphatic tissue for the drug-loaded pad may be lymphatic tissue that drains the primary tumor or involves tumor spreading to a distant site (e.g. intraperitoneal implantation may target retroperitoneal and mediastinal lymph nodes; intrapleural implantation may target both ipsilateral side and contralateral side mediastinal lymph nodes). The drug-loaded pad can be applied following lymphadenectomy (surgical removal of lymph nodes) or lymph node sampling.

The location where the drug-loaded pad is implanted may be anywhere lymphatic tissue exists. Examples include intraperitoneal space (e.g. for ovarian cancer, colorectal cancer, gastric cancer, hepatobiliary cancer, pancreatic cancer, etc.), intra-abdominal cavity, intrapleural space (e.g. for lung cancer, esophageal cancer, mediastinal tumors, etc.), axillary space (e.g. for breast cancer), mastectomy pocket, pelvic cavity (e.g. for prostate cancer, bladder cancer, etc.), subcutaneous space for skin cancer (e.g. melanoma), or other anatomical or surgical compartment (e.g. for head and neck cancers). After the drug-loaded pad is implanted, it degrades and releases the drug particles from the bulk matrix. The drug particles are taken up by the target lymphatic tissue. The treatment may further comprise applying radiation therapy to the site of implantation of the drug-loaded pad or to the lymphatic tissue where the drug particles are absorbed. The treatment may further comprise administering systemic therapy.

The drug-loaded pad may adhere to the target lymphatic tissue. Degradation of the bulk matrix of the drug-loaded pad promotes release of the drug particles. The drug-loaded pad is designed to slowly degrade after implantation. For example, ≥90% of the mass of the drug-loaded pad may be degraded away by 75 days after implantation. In other examples, ≥90% of the mass of the drug-loaded pad may be degraded away by 45, 90, 120, or 150 days after implantation.

Relatively little of the drug released from the drug-loaded pad enters into the systemic circulation of the patient. The highest blood plasma concentration after implantation of the drug-loaded pad (e.g. after 72 hours) could be ≤15 ng/mL, or ≤10 ng/mL. The amount of drug in tissue surrounding the target lymphatic tissue (e.g. surrounding arteries) could be 2-10 times less than the amount in the target lymphatic tissue.

### BRIEF DESCRIPTION OF THE DRAWINGS

FIG. 1 shows an example drug-loaded pad of the invention.

### DETAILED DESCRIPTION OF EXAMPLE EMBODIMENTS

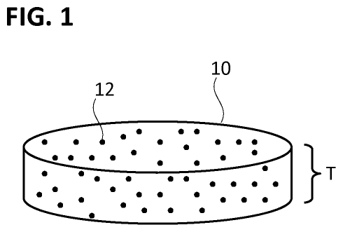

Drawings are provided to help understand the invention and illustrate specific representative examples. The drawings herein are not necessarily made to scale or actual proportions. For example, the size of components may be adjusted to accommodate the page size. FIG. 1 shows an example drug-loaded pad of the invention. The drug-loaded pad 10 has a circular shape (resembling a wafer) and contains numerous biodegradable drug microspheres 12 dispersed within a biodegradable carrier matrix. “T” represents the thickness of the drug-loaded pad.

### Experimental Work

Preliminary Implant Design (Strips Batch-1). Prototypes of the drug-loaded implants of this invention were made as rectangular strips. These strips were made of a soft collagen matrix containing PLGA-paclitaxel microspheres. The amount of paclitaxel in each strip was 14.5 mg. The size of the strip was 50 mm (length)×25 mm (width)×5 mm (thickness). The entire strip weighed 258 mg, with paclitaxel 14.5 mg (5.6 wt %, as PLGA-paclitaxel microspheres), and collagen 150 mg (58.1 wt %).

Toxicology Study #1, Intrapleural Implantation. This study was performed in 31 beagle dogs (16 male, 15 female). The strip was implanted into the mediastinum through the left side of the pleural cavity of the dogs. To vary the dose amount, the strip samples were trimmed prior to implantation. The amount of trimming ranged from none (fully intact, giving a dose of 14.5 mg) to 1/64 size (giving a dose of 0.22 mg). Also, some dogs received multiple strips with further trimming. This resulted in dosing amounts of 36 and 72.5 mg. Controls were sham surgery (surgery only with no implants) and placebo implants (collagen matrix with blank PLGA poly [lactide-co-glycolide] microspheres). The animals were followed for one to four weeks after implantation of the single dose.

The major toxic effect was pleural effusions, which was dose dependent. Paclitaxel doses in the range of 0.22-7.25 mg did not cause significant pleural effusion. Thus, a possible safe unit dose range is about 0.22-7.25 mg of paclitaxel for beagle dogs. Dosing at 10.9 mg caused significant pleural effusion and respiratory compromise after two weeks. But those dogs later showed signs of recovery by four-week follow-up. Thus, we determined that the highest non-severely toxic dose (HNSTD) was 10.9 mg/dog through intrapleural implantation. HNSTD is the dose at which toxicity is observed but recovery from the toxic effect was possible. Thus, a possible acceptable dose range could be about 0.22-11 mg/dog. By extrapolation (see below), the equivalent dose in humans would be about 1-33 mg dosing through the pleural cavity.

Toxicology Study #2 (performed to GLP standards). This study used the rectangular strips (Batch-II) of the same size as described above, except each sample contained 15 mg paclitaxel (8.4 wt % as PLGA-paclitaxel microspheres) and collagen 75 mg (41.8 wt %). Each implant sample was cut into four equal pieces for dosing (3.75 mg/piece). In four Beagle dogs, the cut samples were implanted into the mediastinum of the left pleural cavity via left anterior thoracotomy (single dose). Two dogs (one male and one female) received two pieces of the cut implant samples giving 7.5 mg of paclitaxel. Two dogs (one male and one female) received three pieces of the cut implant samples giving 11.25 mg of paclitaxel. The animals were followed for 19 days after the implantation.

Both the 7.5 mg/animal and 11.25 mg/animal doses resulted in pleural effusion caused by local tissue inflammation adjacent to the implant site including lung, pericardium, and thymus. Respiratory symptoms related to these toxic effects were observed around two weeks post-implant. From this result, the HNSTD (highest non-severely toxic dose) was 11.25 mg/animal.

Further Implant Design (Wafers). Experimental samples of the implant invention were made with an alternate design, designated as SMS-001. The following describes the composition of the experimental samples, each unit containing 1 mg paclitaxel. The drug was made into microspheres of 1-5 μm size. The drug microspheres were made of paclitaxel and poly(lactide-co-glycolide) (PLGA), serving as a sustained-release matrix for the drug vehicle. The carrier formulation was made of collagen and polysorbate 80 (Tween 80) as a solubilizing agent. The drug microspheres were mixed into the carrier formulation. The samples were formed into circular wafers.

The final composition of the wafer, as a percentage of wafer weight, were as follows: the drug constituted 6.7 wt % of the wafer, and the carrier constituted about 55.4 wt % of the wafer. The drug wafers were soft and spongy. The circular wafers were 18 mm in diameter and 4 mm in thickness. When implanted into the body, the wafer becomes a semi-solid gelatinous material that self-adheres to the implantation site.

Toxicology Study #3, Intrapleural Implantation of SMS-001. This toxicology study of the SMS-001 wafer implants was performed in 18 Beagle dogs. The wafers were implanted into the mediastinum within the left side of the pleural cavity of the dogs. To vary the dose amount, different numbers of SMS-001 wafer units (each containing 1 mg of paclitaxel) were implanted in the dogs in nine groups (n=2 for each group). The number of wafers implanted in the nine groups were 1, 4, 5, 7, 8, 9, 10, 11, and 12 wafers. This corresponded to dosing amounts of 1 mg, 4 mg, 5 mg, 7 mg, 8 mg, 9 mg, 10 mg, 11 mg, and 12 mg of paclitaxel, respectively. The animals were monitored for up to four weeks after wafer implantation.

Focal pulmonary pathologic findings were detected microscopically in the area where there was direct contact with the drug wafers. At the higher dose range of 8-11 mg paclitaxel (8-11 wafer units), toxic effects (at both gross and microscopic levels) were found in the experimental animals. All the animals receiving this higher dose range (8-11 mg) developed bilateral pleural effusions and lung injuries.

Necropsy findings revealed that paclitaxel doses of greater than 8 mg (8 wafers) resulted in pleural effusions and focal lung injuries where the wafer had direct contact with the tissue. The highest dose of 12 mg caused severe acute respiratory distress. At the lower dose range of 1-7 mg paclitaxel (1-7 wafer units), all the animals tolerated the treatment well without developing pleural effusion. Thus, a possible safe dose range could be about 1-7 mg/dog. By extrapolation (see below), the equivalent dose in humans would be about 3-21 mg.

Drug Distribution Study. A drug distribution study of the experimental implants was performed in thirty-six Beagle dogs as part of Toxicology Study #5 (see below). The following amounts of paclitaxel were administered to the dogs as a single dose: 1 mg (single drug wafer), 3 mg (three drug wafers), and 10 mg (ten drug wafers). The drug wafers were implanted in the left intrapleural cavity of the dogs.

A total of 9 tissue preparations, including pleural fluid, were analyzed for paclitaxel. As expected, levels of paclitaxel correlated with proximity to the location of the wafer implant. The mediastinal lymph nodes (at targeted site) had the highest level of paclitaxel, followed by lung tissue on the left and right sides. Heart, aorta, and pulmonary artery showed significantly lower drug levels. In general, ipsilateral (left) mediastinal lymph nodes had higher paclitaxel levels than those on the contralateral side.

Thus, the experimental implants successfully performed targeted drug delivery to lymph nodes. This allows for high concentration drug exposure to targeted lymph nodes without unnecessary drug exposure and associated drug toxicities to surrounding tissues and organs.

The total paclitaxel concentrations in pleural fluid in animals treated with 3 mg and 10 mg drug loading were 254 ng/mL and 303 ng/mL respectively at day. 15. At day 71, the paclitaxel concentration was 272 ng/mL in the 10 mg drug group. This was approximately 1,000 times lower than levels cited from clinical studies of malignant pleural effusion treated with intrapleural paclitaxel (with dosing at 120 mg/m² body surface area).

Toxicology Study #4 (performed to GLP standards). This study used the circular wafer implants described above. These wafer implant samples contained 1 mg paclitaxel/unit. In six Beagle dogs, the samples were implanted onto the mediastinal lymphatic tissue within the left pleural cavity through a left anterior thoracotomy (single dose). Two dogs (one dog/sex) received three wafer implants giving 3 mg of paclitaxel (3 wafers). Two dogs (one dog/sex) received 10 wafer implants giving 10 mg of paclitaxel. The other two dogs were controls (placebo or blank wafers). The animals were followed for 43 or 71 days after implantation. The dogs had placebo treatment and the dogs that received the 3 mg dose of paclitaxel had no pleural effusion. One of the dogs that received the 10 mg dose of paclitaxel had minimal (26 mL) pleural effusion found during necropsy.

Toxicology Study #5 (performed to GLP standards). This study used the circular wafer implants described above. These wafer implant samples contained 1 mg paclitaxel/unit. In 60 Beagle dogs, the samples were implanted into the mediastinum within the left pleural cavity through a left anterior thoracotomy (single dose). One group received a single wafer implant giving 1 mg of paclitaxel. Another group received three wafer implants giving 3 mg of paclitaxel. Another group received 10 wafer implants giving 10 mg of paclitaxel. The other groups were controls (sham surgery and placebo blank wafers). The animals were followed for up to 71 days after implantation. Based on a standard intravenous dose of paclitaxel for Beagle dogs at 165 mg/m² and an average body surface area (BSA) of 0.37 m², the proposed doses of 1 mg, 3 mg, and 10 mg for the paclitaxel wafer correspond to approximately 1.6%, 5%, and 16%, respectively, of a single standard intravenous dose. Ultrasound examination of the thoracic cavity was performed to detect any pleural effusion. The 1 mg/animal group had no pleural effusion. One dog in the 3 mg/animal group developed a pleural effusion (130 mL volume on necropsy). A large volume of pleural effusions was detected in all animals of the high dose group (10 mg/animal) on day 15. The volume of pleural effusion decreased over the subsequent 42 days. On day 71, the pleural effusions were absorbed and were undetectable during ultrasound examination.

In the 3 mg/animal and 10 mg/animal groups, the necropsy examination on day 15 revealed local inflammation limited to organs and tissues adjacent to the implant site. This included dark-red discoloration of the lungs and adhesions on the lungs, thymus, and pericardium. On day 71 the necropsy examination, the lung adhesions had reduced in severity compared to day 15.

On microscopy, in the 3 mg/animal and 10 mg/animal groups, there was mild to moderate inflammation at the lungs, pericardium, thymus, diaphragm, and upper mediastinum lymph nodes. In the 10 mg/animal group, there was fibrosis in the lungs and focally in pulmonary pleura. These problems occurred between days 3 and 15 gradually reduced in severity over subsequent days. The drug microspheres are microscopically evident in the ipsilateral side mediastinal lymph nodes as well as that of the contralateral side mediastinal lymph nodes, indicating an effective targeted delivery of drug microspheres to both regional and remote lymphatic tissue.

Total paclitaxel levels were measured in the heart, aorta, left and right lungs, left and right-side mediastinal lymph nodes, pulmonary artery, and pleural effusions. For pleural effusions, free non-protein-bound paclitaxel was separately measured. As expected, different sites had different levels of paclitaxel that was correlated with their physiological connection to the site where the wafer was implanted.

The mediastinal lymph nodes (targeted site) had the highest level of total paclitaxel (therapeutic level), followed by the left side and right-side lung tissues. In contrast, the heart, aorta, and pulmonary artery showed significantly lower levels of paclitaxel. Relatively low levels of paclitaxel were detected in pleural effusions from mid-dose (3 wafers implanted, 3 mg) and high dose (10 wafers implanted, 10 mg) treated animals. Notably, the majority of pleural effusion samples contained unquantifiable levels of free non-protein-bound paclitaxel.

Conclusions of Toxicology Study #5. At a dose of 1 mg/animal, there were no adverse effects resulting from the implant. At a dose of 3 mg/animal and 10 mg/animal, adverse effects were limited to the implant site and adjacent tissues/organs. The pathologic findings reduced in severity over the post-surgical recovery period. The most noticeable clinical toxic findings were pleural effusion (in the absence of chest tube drainage) and its associated manifestations such as tachycardia and tachypnea, especially at the dose level of 10 mg/animal. However, there was no compromise in lung volume or respiratory function. Because patients will have a chest tube in place as part of routine post-operative management to drain the pleural space or thoracic cavity, the potential risks of developing excessive pleural effusions appear manageable.

As monitored by ultrasound, the pleural effusions were significantly absorbed from day 43 onward, and almost completely gone by day 71. This study found that the HNSTD (highest non-severely toxic dose) was 10 mg/animal. This study also showed that the wafer implant materials were mostly absorbed by day 71. Local tissue reactions were minimal across all implant groups. There was no detectable drug in plasma at 1 mg/animal; occasionally low levels detected at 3 mg/animal; and minimal and short-term (up to 96 hours) systemic exposure at 10 mg/animal after a single implant dose. Therefore, systemic toxicities associated with conventional intravenous administration of paclitaxel were avoided.

Single Dose Pharmacokinetic Study of SMS-001 Wafer in Sprague-Dawley Rats by Intraperitoneal Implantation. Twenty-four healthy female Sprague-Dawley rats (body weight 180-220 g) were treated with the intraperitoneal implantation of one SMS-001 wafer (paclitaxel 1 mg). The animals were euthanized, and plasma as well as the following tissue samples were collected at various time points (Day 1, Day 3, Week 1, Week 2, Week 4, Week 6, Week 8, and Week 10): left ovary, right ovary, celiac lymph nodes, pelvic lymph nodes, mediastinal lymph nodes, omentum, and mesentery.

Results demonstrate that SMS-001 generates significant paclitaxel exposure in both ovaries, regional lymph nodes (celiac, pelvic), remote lymph nodes (mediastinal), and lymphoid tissues (omentum, mesentery). Paclitaxel remained at significant levels in the targeted lymphatic and lymphoid tissues for an extended duration (for at least 8 weeks). In contrast, plasma paclitaxel levels were minimal or undetectable after 24 hours, indicating that systemic toxicities associated with paclitaxel can be effectively avoided.

No encapsulation of the implant was found at the local implantation site. No obvious adhesions were identified. The drug microspheres were microscopically identified in both regional lymphatic and lymphoid tissue, as well as remote mediastinal lymph nodes.

The results suggest that SMS-001 has the potential for localized application during cancer surgery to target lymphatic metastases from various malignancies within the peritoneal and pelvic cavities, including ovarian, gastrointestinal, colorectal, hepatobiliary, pancreatic, kidney, bladder, and prostate cancers, among others. Since paclitaxel is a radiation sensitizer, the local implantation of SMS001 during cancer surgery may enhance therapeutic effect in controlling lymphatic metastasis, especially when combined with postoperative radiation therapy.

Pharmacokinetic Study of SMS-001 Wafer (Paclitaxel 1 mg) Through Left Armpit (Axillary Space) Implantation in Rats. Twenty-two healthy female Sprague-Dawley rats received implantation of one SMS-001 wafer to the left side armpit. The animals were euthanized, and plasma as well as the following tissue samples were collected at various time points (Day 3, Week 1, Week 2, Week 4, Week 8, Week 10 and Week 12): left axillar lymph nodes, right axillar lymph nodes, and mediastinal lymph nodes.

The paclitaxel exposure in the left side axillar lymph nodes was significantly high for at least 12 weeks. The paclitaxel was also measurable on the contralateral side axillar lymph nodes and mediastinal lymph nodes during the experimental period. Systemic paclitaxel exposure was very minimal and transient, indicating systemic toxicities of paclitaxel can be avoided. The results suggest that SMS-001 has the potential to target lymphatic metastases in breast cancer, either during diagnostic procedures such as lymph node biopsy or in conjunction with mastectomy.

Extrapolation to Human Dosing. For drug-releasing implants, there is no existing dose extrapolation formula for calculating a human dose from animal data. Moreover, conventional body surface area is not suitable for extrapolating the dose because the systemic drug distribution is minimal. An alternate extrapolation technique is needed. The above dog study demonstrates that drug exposure is mainly limited to the thoracic cavity. Thus, comparing the pleural surface area in the experimental animals to the reported human pleural surface area would be a suitable technique for extrapolating the animal dose to an equivalent human dose.

The average pleural surface area of an adult Beagle dog (7-9 kg) is 779 cm²; whereas the average pleural surface area of an adult human is reported to be 2,120 cm² for a body weight of about 62 kg and height of 167 cm. Thus, the human pleural surface area is about three times that of the Beagle dog. Because the above studies indicate that 1-3 mg of paclitaxel in the form of microparticles embedded in a biodegradable carrier matrix is a safe dose range for Beagle dogs (in the pleural cavity), the equivalent safe dose range of paclitaxel (by drug release into a localized area) in humans would be 3-9 mg.

The conventional dose of paclitaxel in humans by intravenous administration is 135-175 mg/m² body surface area (BSA). Furthermore, a similar dose range is applied by intrapleural administration for controlling malignant pleural effusions in humans. Based on the average adult human BSA of 1.7 m², the proposed dose of 3-9 mg paclitaxel is approximately only 1-4% that of a standard intravenous or intrapleural dose.

For intraperitoneal implantation of the drug loaded pad, the drug exposure is mainly localized to the peritoneal cavity, we can estimate the human equivalent dose (HED) based on the peritoneal surface area (PSA) ratio between species. This method follows a similar logic used in pleural surface area scaling.

$$HED = Animal Dose \times ( Human Peritoneal Surface area / Animal Peritoneal Surface Area )$$

$$Animal Dose ( mg ) = tested paclitaxel dose in Sprague - Dawley rats ( 1 mg / animal )$$

$$Human Peritoneal Surface Area ( PSA ) = \sim 14 , 000 {cm}^{2} ( for a 70 kg human )$$

$$Rat Peritoneal Surface Area ( PSA ) = \sim 225 {cm}^{2} ( for a weight of 250 g )$$

The extrapolated human intraperitoneal implantation dose is estimated to be 62 mg, based on the 1 mg SMS-001 (paclitaxel) dose administered in Sprague-Dawley rats. However, higher doses may not be necessary for localized drug delivery, provided that effective drug exposure is achieved in the target lymphatic tissue. Therefore, a conservative dose range of 1-60 mg is proposed to balance safety and efficacy. However, other calculations indicate that even larger doses are possible for human use when the wafer is administered into different body compartments.

Subsequently, the investigational new drug SMS-001 wafer (paclitaxel 1 mg) for intrapleural implantation was approved by the US FDA for a First-in-Human clinical trial. This treatment aims to control lymphatic metastasis in non-small cell lung cancer patients undergoing cancer surgery. A 65-year-old gentleman diagnosed with non-small cell lung cancer was treated with SMS-001 intraoperatively when he underwent surgical resection of his left upper lobe lung cancer. Three SMS-001 wafers were implanted onto the mediastinal lymphatic tissue (lymph node fossa) where the suspicious lymph nodes were identified and surgically removed. The patient tolerated the procedure and treatment well. The postoperative recovery was uneventful. So far (3 months after the wafer implantation), there is no SMS-001 related adverse event reported. More patients are being enrolled into the study.

### Additional Embodiments of the Invention

In another embodiment (targeted application), the invention is a method for treating cancer. The method comprises applying the biodegradable drug-loaded pad directly to lymphatic tissue during a surgical procedure. The drug-loaded pad releases the drug to eliminate residual tumor cells while preserving lymphatic function and minimizing surgical complications.

In another embodiment (intraoperative use), the invention is a method of reducing surgical complications during lymphadenectomy. The method comprises applying the biodegradable drug-loaded pad to lymphatic tissue intraoperatively. The drug-loaded pad conforms to the tissue and self-adheres without sutures. This provides localized sustained drug release to reduce the need for extensive lymph node dissection.

In another embodiment (prevention of complications), the invention is a method for minimizing lymphedema in a patient undergoing cancer surgery. The method comprises implanting the biodegradable drug-loaded pad onto lymphatic tissue. The drug-loaded pad provides localized chemotherapy to treat potential tumor metastases while preserving lymphatic pathways.

In another embodiment (combination treatment), the invention is a method of treating cancer metastasis. The method comprises surgically resecting a primary tumor, followed by the application of the biodegradable drug-loaded pad to regional lymphatic tissue. The drug-loaded pad releases the drug in a controlled manner to prevent lymphatic tumor spread and enhance postoperative recovery.

In another embodiment (specific anatomical use), the invention is a method of treating mediastinal lymphatic metastases. The method comprises applying the biodegradable drug-loaded pad containing paclitaxel to lymphatic tissue during a thoracoscopic procedure. The drug-loaded pad releases the paclitaxel over a duration of 1-3 months and minimizes systemic toxicity.

In another embodiment, the invention delivers the drug into lymphatic tissue at a localized concentration of less than 5% of the standard systemic dose. In another embodiment, the invention delivers the drug with a controlled-release profile that enables therapeutic levels of the drug into the lymphatic system with a dose that is less than 5% of the standard intravenous administration. In another embodiment, the invention exposes the targeted lymphatic tissue to therapeutic levels of the drug while mitigating the risks of off-target toxicity by significantly reducing the overall dose amount of the drug.

### Potential Advantages of the Invention

As mentioned above in the Background section, metastatic cancer cells often spread from the primary tumor to other parts of the body through the lymphatic system. Managing lymphatic spread of cancer is important for treatment and staging. Lymphadenectomy is a commonly performed procedure that removes lymph nodes to control and assess cancer spread. As mentioned above, the implantation of the drug-loaded pad could be performed in combination with lymphadenectomy. As used herein, the term “lymphadenectomy” encompasses lymph node sampling. Lymphadenectomy is a critical component of surgical oncology for staging, prognosis, and treatment. As further explained below, use of this invention releases the drug directly to lymphatic tissue for high effectiveness, thereby making lymphadenectomy simpler and safer.

Lymphadenectomy is a complex and challenging surgical procedure that comes with serious risks and complications. There are technical challenges such as adequate sampling, which requires a balance between removing enough lymph nodes for accurate staging and avoiding excessive lymph node dissection. There is also the problem of intraoperative identification of lymph nodes by techniques (such as dye injection or radiotracer mapping) which are time-consuming and require specialized expertise. There is further the problem of limited visualization of the lymph nodes when using minimally invasive surgical techniques (such as laparoscopic or robotic surgery). The limited tactile feedback and reduced visibility in such situations can complicate lymph node identification.

Conventional lymphadenectomy is further limited by oncological considerations. For example, small metastatic deposits (micrometastasis) may be missed, even despite extensive sampling. This could result in understaging of cancer. In another example, extensive lymphadenectomy may not always improve survival outcomes and have the opposite effect instead (i.e. increasing morbidity). This raises the concern of overtreatment in certain cases. Involvement of sentinel lymph nodes presents yet another example of oncological considerations that limit lymphadenectomy. Although identifying sentinel lymph nodes is crucial, misidentification or failure to locate sentinel lymph nodes can compromise staging accuracy. By delivering the drug directly to the lymphatic system, this invention could supplement surgical removal of visibly affected lymph nodes by targeting micrometastasis within lymph nodes and preventing further spread.

Conventional lymphadenectomy is also subject to pathological limitations. There is often variability in the pathological assessment and reporting of removed lymph nodes, which can affect the accuracy of cancer staging. Failure to retrieve an adequate number of lymph nodes for pathological examination could also lead to inaccurate staging of cancer (e.g. understaging in colon cancer).

Conventional lymphadenectomy is also limited by patient-specific factors. Many patients are obese, and adipose tissue can obscure lymph nodes, making them harder to locate and increasing operative difficulty. Previous surgery or radiation therapy can limit lymphadenectomy. Scar tissue or fibrosis from such prior treatments can obscure lymphatic anatomy and complicate lymph node dissection.

Conventional lymphadenectomy also increases post-operative risks. Surgical disruption of lymphatic flow can cause fluid collections (seromas) or increase the risk of infections. Moreover, aggressive lymphadenectomy may result in chronic pain, mobility problems, or other quality-of-life problems.

Conventional lymphadenectomy also requires specialized techniques and expertise. Lymphadenectomy is highly dependent on the technology available to the surgeon. Advanced imaging and intraoperative tools (e.g. fluorescence imaging or robotic assistance) may be employed, which require specialized training and are not always universally available. Moreover, expertise in sentinel lymph node biopsy and advanced lymphadenectomy techniques varies among surgeons, which impact patient outcomes.

Lymphadenectomy requires the careful balancing of risks and benefits. The therapeutic value of extensive lymphadenectomy remains controversial for many cancers because it may actually increase the risk of complications without significantly improving survival outcomes. In this regard, this invention could work synergistically with lymphadenectomy to result in various benefits, such as making less aggressive surgery possible, having fewer complications, eliminating micrometastases in lymph nodes, preserving lymphatic function, enhancing local regional control of the cancer, reducing systemic toxicity, preventing the spread of the cancer cells during surgery, reducing recurrence in high risk cancers, minimizing the need for postoperative adjuvant therapy, and improving long term survival.

The anatomical structure and drainage patterns of the lymphatic system are highly complex and vary between individuals. This can complicate identifying and removing lymph nodes. Also, lymph nodes are often located near critical structures such as major blood vessels, nerves, and organs. This increases the risk of inadvertent injury while performing lymphadenectomy. For example, injury to a major blood vessel during dissection of lymph nodes can cause significant blood loss or blood clots. The focused drug delivery implemented by this invention could help with minimizing lymph nodes dissection near such delicate structures, thereby having a protective effect against injury to such delicate structures.

Preserving lymphatic function is an important consideration in lymphadenectomy. Because removal of lymph nodes can disrupt lymphatic drainage, lymphedema could also be a complication that severely affects quality of life. This is particularly a problem in the limbs or adjacent regions (e.g. lymphedema in the arm after mastectomy). The focused drug delivery implemented by this invention would address metastatic disease pharmacologically. This could help with minimizing lymph node dissection (and the resulting disruption to lymphatic function). That is, by permitting the possibility of less aggressive surgery and reducing the number of lymph nodes that need to be removed, this invention could help to reduce lymphedema caused by lymphadenectomy.

Even carefully performed lymph node dissection can injure nearby nerves (e.g. damage to the recurrent laryngeal nerve causes hoarseness). Such damages could also result in sensory (e.g. chronic pain) or motor deficits. Again, the focused drug delivery implemented by this invention could help with minimizing lymph node dissection near such delicate structures.

This invention could also help address the challenges of having local and regional control of cancer. Regional recurrence is a significant problem in the management of cancer, particularly in cancers with extensive lymphatic involvement and high propensity for lymphatic spread (e.g. lung, colorectal, hepatobiliary, and pancreatic cancers). By specifically targeting and acting directly within the lymphatic system, this invention could improve local and regional control of the disease. This could significantly reduce recurrence rates and improve long-term outcomes in these cancers.

This invention could also work to reduce systemic toxicity of conventional systemic chemotherapy, which is associated with off-target toxic effects. Such toxic effects limit the dosage amount of the chemotherapy (i.e. dose-limiting toxicities). The trans-lymphatic drug delivery provided by this invention ensures localized drug action within the lymphatic system, thus reducing systemic drug exposure and its associated toxic side effects.

This invention could also work to prevent the spread of tumor cells during surgery. Surgical manipulation of tumors and lymph nodes can inadvertently release tumor cells into lymphatic and vascular channels, thereby increasing the risk of cancer metastasis. In perioperative use, the drug-loaded pad of this invention could act as a barrier to tumor cell dissemination, thereby reducing the risk of surgical seeding of cancer cells.

This invention could also complement lymphadenectomy with better management of tumor spread, quicker recovery, and fewer complications. Complete removal of all metastatic lymph nodes is often impractical because of anatomic and physiologic constraints. When used as an adjunct to surgery, this invention could treat residual disease in non-resected lymph nodes or lymphatic tissues, thereby providing a more comprehensive therapeutic approach. Moreover, this invention could help minimize the need for post-operative adjuvant therapies. Many patients require systemic adjuvant chemotherapy post-surgery, which may not effectively target lymphatic metastasis. The targeted lymphatic delivery provided by this invention could reduce or replace the need for systemic adjuvant therapies, which could reduce side effects and improve patient compliance.

In summary, lymphatic metastasis is an important driver of cancer progression and mortality. By addressing lymphatic metastasis more effectively than surgery or systemic therapy alone, this invention could improve disease-free survival and overall survival in cancer patients. Moreover, this unique and transformative invention could serve as a platform technology to address the challenges of lymphatic metastasis, which is a common feature across various cancers. As a platform technology, this invention could address the current limitations in specifically targeting the lymphatic system. This invention could possibly reduce or replace the need for systemic adjuvant therapies, which could lead to fewer side effects and better patient compliance. Because the trans-lymphatic mechanism of action in this invention could be adapted to other cancers, this invention could be a versatile tool in surgical interventions for diverse cancers.

The foregoing description and examples merely illustrate the invention and are not intended to be limiting. Each of the disclosed aspects and embodiments of the invention may be considered individually or in combination with other aspects, embodiments, and variations of the invention. Also, unless otherwise specified, the steps of the methods of the invention are not limited to any particular order of performance. Persons skilled in the art may perceive modifications to these embodiments that incorporate the spirit and substance of the invention. Such modifications are within the scope of the invention.

Any use of the word “or” herein is intended to be inclusive and is equivalent to the expression “and/or,” unless the context clearly indicates otherwise. As such, for example, the expression “A or B” means A, or B, or both A and B. Similarly, for example, the expression “A, B, or C” means A, or B, or C, or any combination thereof.

1. A drug-loaded pad comprising:
a bulk matrix comprising a biodegradable polymer;
biodegradable drug particles carried by the bulk matrix, wherein the drug particles comprise a drug, wherein the drug is paclitaxel or docetaxel or cabazitaxel;
wherein the drug constitutes 3-40 wt % of the drug-loaded pad.

2. The drug-loaded pad of claim 1, having a total mass of ≥10 mg.

3. The drug-loaded pad of claim 1, containing 0.2-12 mg of the drug.

4. The drug-loaded pad of claim 1, wherein the drug particles have a size in the range of 0.5-10 μm.

5. The drug-loaded pad of claim 1, wherein the drug particles have a size in the range of 10-500 nm.

6. The drug-loaded pad of claim 1, wherein the bulk matrix constitutes 30-75 wt % of the drug-loaded pad.

7. The drug-loaded pad of claim 1, wherein the drug-loaded pad has a thickness of 2-8 mm.

8. The drug-loaded pad of claim 1, wherein the drug constitutes 5-25 wt % of the drug-loaded pad.

9. The drug-loaded pad of claim 1, containing ≤8 mg of the drug.

10. A method of treating cancer in a patient, comprising:
having a drug-loaded pad of claim 1;
implanting the drug-loaded pad onto a target lymphatic tissue.

11. The method of claim 10, wherein the drug-loaded pad is implanted without sutures or glue.

12. The method of claim 10, further comprising performing a surgery for either diagnostic or tumor resection surgery to resect a primary tumor, wherein implanting the drug-loaded pad is performed intraoperatively during the surgery, and the target lymphatic tissue drains the primary tumor.

13. The method of claim 10, further comprising performing lymphadenectomy in conjunction with implanting the drug-loaded pad.

14. The method of claim 10, wherein the drug-loaded pad is shapeable and conforms to the target lymphatic tissue.

15. The method of claim 10, wherein the amount of the drug given to the patient via the drug-loaded pad is in the range of 2-15 mg.

16. The method of claim 15, wherein the amount of the drug given to the patient via the drug-loaded pad is in the range of 3-9 mg.

17. The method of claim 10, wherein the amount of the drug given to the patient via the drug-loaded pad is in the range of 1-60 mg.

18. The method of claim 17, wherein the amount of the drug given to the patient via the drug-loaded pad is in the range of 3-21 mg.

19. The method of claim 10, wherein ≥90% of the drug-loaded pad is degraded away by 75 days after implantation.

20. The method of claim 10, wherein a highest blood plasma concentration of the drug after implantation of the drug-loaded pad is ≤15 ng/ml.
